(19) United States
(12) Patent Application Publication
(10) Pub. No.: US 2012/0329761 A1
(43) Pub. Date: Dec. 27, 2012

(54) USE OF TIGECYCLINE FOR TREATMENT OF CANCER

(75) Inventors: Aaron D. Schimmer, Thornhill (CA); Marko Skrtic, Toronto (CA)

(73) Assignee: UNIVERSITY HEALTH NETWORK, Toronto, ON (CA)

(21) Appl. No.: 13/582,842

(22) PCT Filed: Mar. 10, 2011
(86) PCT No.: PCT/CA11/00258

§ 371 Date: Sep. 5, 2012

Related U.S. Application Data
(60) Provisional application No. 61/312,410, filed on Mar. 10, 2010.

(51) Int. Cl. A61K 31/65; C12Q 1/68; A61P 35/02; C12N 5/09; C07C 237/26; A61P 35/00

(52) U.S. Cl. 514/152; 552/205; 435/375; 435/6.11

(57) ABSTRACT

Cancer stem cells exhibit different metabolic profiles from other cancer cells, such that they do not readily respond to treatment using conventional chemotherapeutic agents. Studies disclosed herein now demonstrate that the glycylcycline antibiotic tigecycline (a tetracycline derivative) exhibits anti-cancer activity, including activity against cancer stem cells. This anti-neoplastic activity appears to be due to inhibition of mitochondrial protein synthesis in the cancer cells. In preferred embodiments, the cancer to be treated is a hematological cancer, such as leukemia, lymphoma or myeloma.

### RELATED APPLICATIONS

This is a Patent Cooperation Treaty Application which claims the benefit of 35 U.S.C. 119 based on the priority of corresponding U.S. Provisional Patent Application No. 61/312,410 filed Mar. 10, 2010, which is incorporated herein in its entirety.

### FIELD OF THE DISCLOSURE

The disclosure relates to methods and compositions for the treatment of cancer and particularly to methods and compositions comprising tigecycline for the treatment of leukemia such as acute myeloid leukemia (AML).

### BACKGROUND OF THE DISCLOSURE

### Cancer Stem Cells

Today's most challenging aspect of cancer therapy is perhaps the cancer stem cell (CSC). Stem cells were first described in 1961 by Till and McCulloch¹, and are generally defined by their potential for self-renewal and differentiation ability into diverse cell types. Cancer stem-cells, which comprise a minority component of tumours, are believed to have the capacity to initiate and sustain the tumourigenic process. It is difficult to eradicate them completely during treatment, and therefore they have become an intriguing target for cancer therapy.

Much of the evidence for the cancer stem-cell hypothesis has come from studies in hematologic malignancies. Studies by Dick and colleagues² found leukemia stem-cells (LSC) in a small compartment from the peripheral blood for Acute Myeloid Leukemia (AML) patients. They were then able to successfully engraft these LSCs into the bone marrow of non-obese diabetic-severe combined immunodeficient (NOD-SCID) mice where these human cells proliferated and disseminated a phenotype similar to that in the original patients. As a result, the current functional standard of a LSC is the successful engraftment into NOD-SCID mice.

Although LSCs have the capacity for self-renewal and differentiation, evidence has shown that a substantial number of LSCs are found in a quiescent G₀ phase³. This could be a possible reason for the failure of chemotherapeutics to eliminate LSCs as they commonly target rapidly cycling populations. Other reasons for LSC resistance to drugs and toxins could be the expression of ATP-associated transporters⁴ and resistance to apoptotic stimuli⁵. Therefore, it would be beneficial to find novel therapeutic compounds that will directly effect the viability of leukemia stem cells.

### SUMMARY OF THE DISCLOSURE

An aspect of the disclosure includes a method of inducing cytotoxicity in a cancer cell comprising contacting the cell with a glycylcycline.

Another aspect of the disclosure includes a method of treating a cancer comprising administering to a subject in need thereof an effective amount of a glycylcycline, such as tigecycline.

A further aspect of the disclosure includes a use of a glyclycycline for treating a cancer such as tigecycline.

In an embodiment, the glycylcycline comprises tigecycline.

In an embodiment, the cancer is a hematological cancer or a solid cancer.

In an embodiment, the hematological cancer is a leukemia. In a further embodiment, the leukemia is acute myeloid leukemia (AML).

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1. Screen in TEX and M9-ENL-1 cells identifies tigecycline with novel anti-leukemia activity. (A) TEX and (B) M9-ENL1 cells were plated in 96-well plates and drugs were added to the wells (5 μL per well) for final concentrations of 10 μM (shown) and 1 μM. Cell growth and viability was measured at 48 hours (M9-ENL-1) and 72 hours (TEX) by MTS assay. Cell viability is shown for each compound as a percent of cells treated with DMSO alone. (C) Leukemia, (D) myeloma, and (E) solid tumor cell lines were seeded in 96-well plates and then treated with increasing concentrations of tigecycline. Cell growth and viability was measured at 72 hours by MTS assay. Cell viability is expressed as mean percentage plus or minus SD (n=3) relative to vehicle-treated cells. (F) Tigecycline displays time-dependent increases in apoptosis in TEX cells, determined by flow cytometry as percentage of cells labeled by Annexin V. (G) TEX cells were seeded in 96-well plates and then treated with increasing concentrations of tigecycline, minocycline and tetracycline. Cell viability was measured at 72 hours by MTS assay. Cell viability is expressed as mean percentage plus or minus SD (n=3) relative to vehicle-treated cells.

FIG. 2. Tigecycline induces cell death and inhibits clonogenic growth in primary AML cells preferential over normal hematopoietic cells. Mononuclear cells from peripheral blood of leukemia patients (blast count >80%) and normal G-CSF expanded donors was treated with increasing concentrations of tigecycline for 48 hours. Cell viability was measured by Annexin-PI flow cytometry staining. Cell viability is expressed as a mean percentage plus or minus SD (n=3) relative to DMSO-treated cells. Mononuclear cells from primary AML patient samples (A, B) and normal peripheral blood cells (C) were plated in methylcelluose with 5 μM of Tigecycline. (D) Colony forming units were counted at 7 days (AML) and 14 days (Normal) post plating. Percent colony formation is represented compared to DMSO-treated plated cells.

FIG. 3. Tigecycline has anti-leukemia activity in vivo. (A, B) Human leukemia (OCI-AML2) were injected subcutaneously into the flank of SCID mice. Seven days after injection, once tumors were palpable, mice were treated with tigecycline (50 mg/kg or 100 mg/kg twice daily by i.p. injection) or vehicle control (n=10 per group). Fourteen days after injection of cells, mice were sacrificed, tumors excised and the volume and mass of the tumors were measured. The tumor mass and the mean volume+SD are shown. Differences in tumor volume and mass were analyzed by an unpaired t-test: * p<0.001. (C) Tumours from two control mice, and three tigecycline-treated mice were excised after 5 days of treatment and total proteins were extracted and analyzed by western blotting for Cox-1, Cox-2, Cox-4, and tubulin. (D) Primary cells from three AML patients were injected intra-femorally into the right femur of female sub-lethally irradiated NOD/SCID mice. Three weeks after injection mice were treated with tigecycline (100 mg/kg by i.p. injection daily) or vehicle control (n=10 per group) for three weeks. Following treatment, human leukemia cell engraftment in the injected right femur was measured by FACS analysis for human CD45⁺CD19⁻CD33⁺ cells. Data represent mean±SD of engrafted human cells. Cells from one patient experiment were used to assess secondary engraftment in a second generation of NOD/SCID mice. Equal numbers of viable leukemia cells from bone marrow of control and tigecycline treated mice were injected into irradiated NOD/SCID mice, which were not treated with tigecycline. Six weeks later, human leukemia cell engraftment in the injected right femur was measured by FACS analysis for human CD45⁺CD19⁻CD33⁺ cells. (* p<0.005, Student's t-test).

FIG. 4. Tigecycline decreases mitochondrial Cox-1 protein levels in TEX cells. (A) Cells from TEX, OCI-AML2 and two primary AML patients were treated with increasing concentrations (2.5 μM and 5 μM) of tigecycline for 36 and 48 hours of tigecycline. Total proteins were extracted and analyzed by western blotting for Cox-1, Cox-2, Cox-4, grp78, XIAP, actin and tubulin. (B) TEX and AML patient cells were treated with increasing concentrations (2.5 μM and 5 μM) of tigecycline. Cox-1, Cox-2, and Cox-4 mRNA expression relative 18S was determined by quantitative RT-PCR. Data is shown as mean±SD. (C) TEX cells were treated with increasing concentrations of tigecycline and chloramphenicol (CAP) for 72 hours. Complex I, II and IV enzyme activity relative to citrate synthase activity was determined as described in materials and methods. (D) Cells from TEX, primary AML patients, and normal donors were treated with increasing concentrations of tigecycline. Mitochondrial membrane potential (Δψ) was determined by staining cells with JC-1 dye, and flow cytometry analysis (Red/Green ratio). Reactive oxygen species generation (ROS) was determined by staining with h2-DCFDA and Dihydroethidium (DHE).

FIG. 5. Mitochondrial characteristics of acute myeloid leukemia cells. (A) Mitochondrial DNA copy number was determined in mononuclear cells from the peripheral blood of primary AML and normal G-CSF mobilized donors. DNA was extracted from cells and real-time PCR was performed for mitochondrial ND1 relative to human globulin (HGB). ND1/HGB ratio is shown relative to cells from one normal G-CSF mobilized donor. (B) Mitochondrial mass was assessed in AML bulk blasts and CD45+/CD34+ cells and compared to CD45+/CD34+ cells from normal G-CSF mobilized individuals. Mitochondrial mass was measured by incubating cells with Mitotracker Green FM dye, and subsequent flow cytometry. Median fluorescence intensity is shown relative to one normal G-CSF mobilized donor. (C) Mitochondrial mass was measured in blasts from eleven AML patients using Mitotracker Green FM method. Cells were then incubated with increasing concentrations of tigecycline for 48 hours. Viability was assessed by Annexin-V/PI staining and subsequent flow cytometry. The correlation between baseline mitochondrial mass and sensitivity to tigecycline at doses of 5 μM and 10 μM is shown.

### DETAILED DESCRIPTION OF THE DISCLOSURE

### I. Definitions

The term “glycylcycline” as used herein means any glycyl derivative of any tetracycline, for example a tert-butyl-glycylamido derivative, and includes any salt forms, such as any pharmaceutically acceptable salt, enantiomer, stereiosomer, solvate, prodrug or mixtures thereof. For example, see^{6,7}. In an embodiment, the glycyl derivative is a tetracycline wherein a glycyl group is attached at the 9 position of the tetracyclic structure.

The term “glycyl” as used herein means a group of the formula:

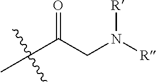

wherein R′ and R″ are independently selected from the group H, C₁₋₂₀alkyl, C₆₋₁₀aryl and C₃₋₁₀cycloalkyl, or R′ and R″ are joined to form, together with the nitrogen to which they are attached, a 3 to 10 membered ring. In an embodiment, one of R′ and R″ is H and the other of R′ and R″ is C₁₋₆alkyl (branched or unbranched).

The term “tigecycline” as used herein means a compound having the structure:

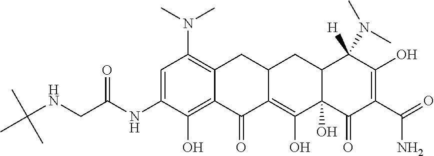

or pharmaceutically acceptable salts, solvates or prodrugs thereof as well as mixtures thereof. Tigecycline can be produced according to methods known in the art for example as described in U.S. Patent Publication Nos.: 2006-0247181, titled “Tigecycline compositions and methods of preparation”; and 2007-0026080, titled “Manufacturing process for tigecycline”.

The term “mixture” as used herein, means a composition comprising two or more compounds. In an embodiment a mixture is a mixture of two or more distinct compounds. In a further embodiment, when a compound is referred to as a “mixture”, this means that it can comprise two or more “forms” of the compounds, such as, salts, solvates, prodrugs or, where applicable, stereoisomers of the compound in any ratio. A person of skill in the art would understand that a compound in a mixture can also exist as a mixture of forms. For example, a compound may exist as a hydrate of a salt or as a hydrate of a salt of a prodrug of the compound. All forms of the compounds disclosed herein are within the scope of the present disclosure.

The term “cancer” as used herein means a metastatic and/or a non-metastatic cancer, and includes primary and secondary cancers. Reference to cancer includes reference to cancer cells.

The term “hematological cancer” as used herein refers to cancers of blood and bone marrow, such as leukemia, multiple myeloma and lymphoma and includes primary and secondary cancers. Reference to hematological cancer includes reference to hematological cancer cells

The term “leukemia” as used herein means any disease involving the progressive proliferation of abnormal leukocytes found in hematopoietic tissues, other organs and usually in the blood in increased numbers. Leukemia includes, but is not limited to, acute myeloid leukemia (AML), acute lymphocytic leukemia (ALL), chronic lymphocytic leukemia (CLL) and chronic myelogenous leukemia (CML).

The term “myeloma” and/or “multiple myeloma” as used herein means any tumor or cancer composed of cells derived from the hematopoietic tissues of the bone marrow. Multiple myeloma is also known as MM and/or plasma cell myeloma.

The term “lymphoma” as used herein means any disease involving the progressive proliferation of abnormal lymphoid cells. For example, lymphoma includes mantle cell lymphoma, Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas.

The term “solid tumour cancer” as used herein refers to a cancer resulting in one or more solid tumours composed of cancer cells and includes, for example, lung cancer, brain (glioblastomas, medulloblastoma, astrocytoma, oligodendroglioma, ependymomas), liver, thyroid, bone, adrenal, spleen, kidney, lymph node, small intestine, pancreas, colon, stomach, breast, endometrium, prostate, testicle, ovary, skin, head and neck, and esophagus.

The term “pharmaceutically acceptable” means compatible with the treatment of animals, in particular humans.

The term “pharmaceutically acceptable salt” means an acid addition salt which is suitable for, or compatible with, the treatment of patients.

The term “pharmaceutically acceptable acid addition salt” as used herein means any non-toxic organic or inorganic salt of any basic compound. Basic compounds that form an acid addition salt include, for example, compounds comprising an amine group. Illustrative inorganic acids which form suitable salts include hydrochloric, hydrobromic, sulfuric and phosphoric acids, as well as metal salts such as sodium monohydrogen, orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary and not exhaustive, and one skilled in the art could prepare other known varieties of prodrugs. Prodrugs may be, for example, formed with available hydroxy, thiol, amino or carboxyl groups. For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in viva Conventional procedures for the selection and preparation of suitable prodrugs are described, for example, in “Design of Prodrugs” ed. H. Bundgaard, Elsevier, 1985.

Where the compounds according to the disclosure possess one or more than one asymmetric centres, they may exist as “stereoisomers”, such as enantiomers and diastereomers. It is to be understood that all such stereoisomers and mixtures thereof in any proportion are encompassed within the scope of the present disclosure. It is to be understood that, while the stereochemistry of the compounds of the disclosure may be as provided for in any given compound shown herein, such compounds may also contain certain amounts (e.g. less than 20%, less than 10%, less than 5%) of compounds having alternate stereochemistry.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “subject” as used herein includes all members of the animal kingdom including mammals, and suitably refers to humans.

The term “inducing cytotoxicity in a cell” as used herein means causing cell damage that results in cell death.

The term “cell death” as used herein includes all forms of cell death including necrosis and apoptosis.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage leukemia can be treated to prevent progression or metastases, or alternatively a subject in remission can be treated with a compound or composition described herein to prevent recurrence. Treatment methods comprise administering to a subject a therapeutically effective amount of a compound described herein and optionally consists of a single administration, or alternatively comprises a series of applications. For example, the compounds described herein may be administered at least once a week. However, in another embodiment, the compounds may be administered to the subject from about one time per three weeks, or about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required. For example, the compounds are administered to the subject in an amount and for a duration sufficient to treat the patient.

As used herein, the term “dosage form” refers to the physical form of a dose for example comprising a compound of the disclosure, and includes without limitation liquid and solid dosage forms including, for example tablets, including enteric coated tablets, caplets, gelcaps, capsules, ingestible tablets, buccal tablets, troches, elixirs, suspensions, syrups, wafers, resuspendable powders, liquids, solutions as well as injectable dosage forms, including, for example, sterile solutions and sterile powders for reconstitution, and the like, that are suitably formulated for injection.

As used herein, the term “effective amount” or “therapeutically effective amount” means an amount effective, at dosages and for periods of time necessary to achieve the desired result. For example in the context or treating a hematological malignancy, an effective amount is an amount that, for example, induces remission, reduces tumor burden, and/or prevents tumor spread or growth compared to the response obtained without administration of the compound. Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “administered” as used herein means administration of a therapeutically effective dose of a compound or composition of the disclosure to a cell either in cell culture or in a patient.

The term “a mitochondrial translated polypeptide” as used herein refers to a polypeptide that is exclusively translated by a ribosome located in a mitochondria.

The term “mitochondrial mass” as used herein refers to the overall number and/or weight of mitochondria in a cell or number of cells. Mitochondrial mass may be determined or characterized, for example, by incubating cells with Mitotracker Green FM dye, subsequently performing flow cytometry, and determining the median fluorescence intensity of the cells. Mitochondrial mass may also be determined or characterized by incubating cells with Mitotracker Green FM dye, subsequently performing confocal scanning laser microscopy, and quantifying the fluorescence levels using an image software, for example ImageJ (see for example Agnello et al. A method for measuring mitochondrial mass and activity. Cytotechnology Vol 56(3):145-149). The mitochondrial mass of a cell or average mitochondrial mass of a number of cells, for example, in a sample taken from a subject with a cancer, can be compared to a mitochondrial mass of a control cell or number of cells in a sample taken for example from a control subject.

The term “control” as used herein refers to a suitable comparator subject, sample, cell or cells such as non-cancerous subject, blood sample, cell or cells from such a subject, for comparison to a cancer subject, sample (e.g. test sample) cell or cells from a cancer subject; or an untreated subject, cell or cells, for comparison to a treated subject, cell or cells, according to the context. For example, a control for comparing mitochondrial mass includes for example non-cancerous cells such as normal CD34+ hematopoietic cells, for example in a blood sample taken from a control subject free of cancer and/or cancer cells known to have low and/or about normal mitochondrial mass. Control can also refer to a value representative of a control subject, cell and/or cells and/or a population of subjects, for example representative of a normal mitochondrial mass.

The term “sample” as used herein refers to any biological fluid comprising a cell, a cell or tissue sample from a subject including a sample from a test subject, i.e. a test sample, such as from a subject whose mitochondrial mass is being tested, for example, a subject with a cancer, wherein the test sample comprises cancer cells, and a control sample from a control subject, e.g., a subject without a cancer, whose mitochondrial mass is being tested. For example, the sample can comprise a blood sample, for example a peripheral blood sample, a fractionated blood sample, a bone marrow sample, a biopsy, a frozen tissue sample, a fresh tissue specimen, a cell sample, and/or a paraffin embedded section. As an example, wherein the cancer is AML, the sample comprises mononuclear cells.

The term “inhibiting a mammalian mitochondrial ribosome in a cell” as used herein means to reduce compared to an untreated cell, interfering with mitochondrial polypeptide translation of mRNA, reflected for example in the steady state level or the amount of translation product produced over a period of time.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

[symbol]

Further, the definitions and embodiments described are intended to be applicable to other embodiments herein described for which they are suitable as would be understood by a person skilled in the art. For example, in the above passages, different aspects of the invention are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### II. Methods and Compositions

Tigecycline, which is for example sold under the brand name Tygacil®, is presently used for the treatment of certain infections. It is demonstrated herein that pharmacologically achievable concentrations of tigecycline are useful for treating cancer and particularly leukemia.

Accordingly, an aspect of the present disclosure includes a method of treating a cancer comprising administering to a subject in need thereof an effective amount of a glycylcycline such as tigecycline. In another aspect, the disclosure includes use of a glycylcycline such as tigecycline for treating a cancer. Another aspect includes use of a glycylcycline such as tigecycline for the manufacture of a medicament for the treatment of a cancer. In yet a further aspect, the disclosure includes a glycylcycline such as tigecycline for use in the treatment of cancer.

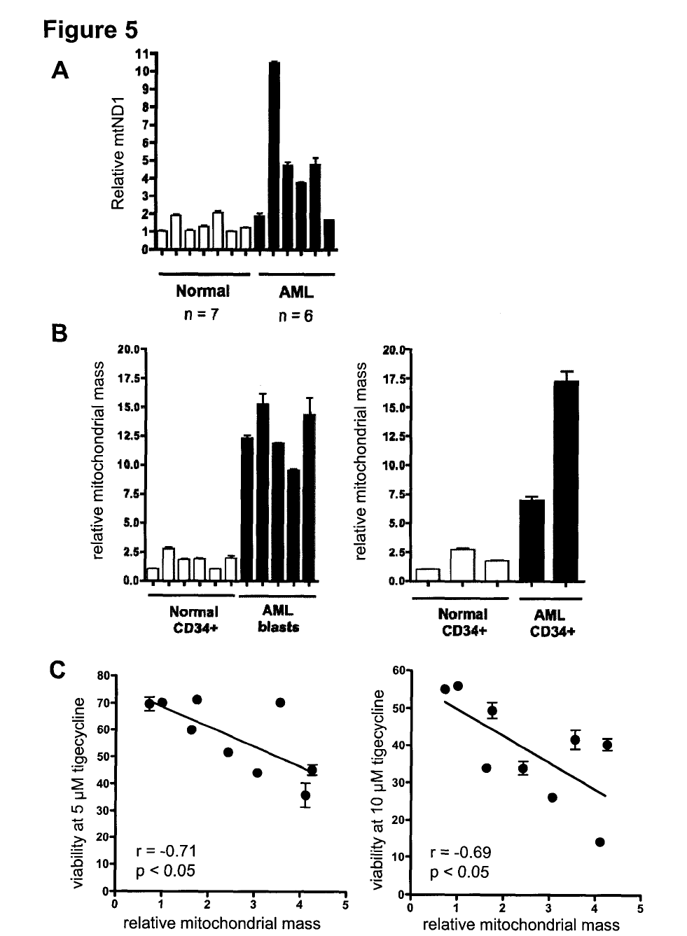

It is also demonstrated herein that tigecycline induced cytoxicity correlates with increasing mitochondrial mass in AML samples. FIG. 5 for example demonstrates that AML cells have an increased mitochondrial mass compared to normal cells and that AML cells with an increased mitochondrial mass are more sensitive to tigecycline compared to cells with a decreased mitochondrial mass.

It is also demonstrated in FIG. 5A that AML samples have increased mitochondrial DNA copy number of ND1 relative to human globin DNA. In FIG. 5A, mitochondrial DNA copy number was determined in mononuclear cells from the peripheral blood of primary AML and normal G-CSF mobilized donors. DNA was extracted from cells and real-time PCR was performed for mitochondrial ND1 relative to human globulin (HGB). ND1/HGB ratio is significantly increased in AML samples.

Accordingly, in an aspect, the disclosure includes a method of identifying a subject likely to benefit from glycylcycline administration comprising: a) obtaining a test sample comprising cancer cells from the subject; b) determining a mitochondrial DNA copy number and/or a mitochondrial mass of the test sample; c) comparing the mitochondrial DNA copy number and/or the mitochondrial mass of the test sample to the mitochondrial DNA copy number and/or the mitochondrial mass of a control, wherein the subject is identified likely to benefit from glycylcyline administration when the test sample has an at least 2 fold increased mitochondrial DNA copy number and/or mitochondrial mass compared to the control. In another aspect, the disclosure includes a method of treating a cancer comprising: a) obtaining a test sample comprising cancer cells from a subject; b) determining a mitochondrial DNA copy number and/or a mitochondrial mass of the test sample; c) comparing the mitochondrial DNA copy number and/or mitochondrial mass of the test sample to a mitochondrial DNA copy number and/or mitochondrial mass of a control, and d) administering a glycylcline to the subject when the mitochondrial DNA copy number and/or mitochondrial mass of the test sample is at least 2 fold increased compared to the mitochondrial DNA copy number and/or mitochondrial mass of the control.

In an embodiment, the gylcyl cycline administered is tigecycline.

In an embodiment, the mitochondrial DNA copy number of a test sample is determined by quantitating a DNA level of a mitochondrial gene such as ND1 and a DNA level of a non-mitochondrial gene (i.e. a nuclear gene) such as human globulin (HGB), which serves as an internal control, and comparing a ratio of the DNA levels of the mitochondrial gene to the non-mitochondrial gene in the test sample to a control. In an embodiment, the method comprises using PCR for example real-time PCR. A person skilled in the art would recognize that a DNA level of any of the genes encoded by the mitochondrial genome. Also, the non-mitochondrial gene can be any suitable nuclear gene such as but not limited to beta-globin, 18S and GAPDH.

A further aspect includes a method of treating a cancer with an at least 2 fold increased mitochondrial DNA copy number and/or mitochondrial mass compared to a control comprising administering to the subject in need thereof, an effective amount of a glycylcycline such as tigecycline.

A further aspect includes use of a gylcylcyline such as tigecyline for treating a cancer with an at least 2 fold increased mitochondrial DNA copy number and/or mitochondrial mass compared to a control. Another aspect includes use of a gylcylcyline such as tigecycline for the manufacture of a medicament for treating a cancer with an at least 2 fold increased mitochondrial DNA copy number and/or mitochondrial mass compared to a control. Yet another aspect includes a gylcylcyline such as tigecycline for treating a cancer with an at least 2 fold increased mitochondrial DNA copy number and/or mitochondrial mass compared to a control. For example, a cancer or cell mitochondrial mass is assessed by taking a biopsy sample e.g. test sample from a subject and determining the mitochondrial mass of the test sample cancer cells using for example a method described herein.

In an embodiment, the cancer and/or cancer cells have at least a 3 fold increase, at least a 4 fold increase and/or at least a 5 fold increase in mitochondrial DNA copy number and/or mitochondrial mass compared to a control.

In another aspect, the disclosure includes a method of inducing cytotoxicity in a cancer cell comprising contacting the cancer cell with a glycylcycline such as tigecycline. The contact is for example under a suitable length of time and under suitable conditions to induce cytotoxicity in the cell. In a further aspect, the disclosure provides use of a glycylcycline such as tigecycline for inducing cytotoxicity in a cancer cell. Another aspect of the disclosure includes use of a glycylcycline such as tigecycline for the manufacture of a medicament for inducing cytotoxicity in a cancer cell. A further aspect provides a glycylcycline for inducing cytotoxicity in a cancer cell. In an embodiment, the cancer cell is in vitro. In an embodiment, the cancer cell is in vivo. In an embodiment, the cancer cell is located in a human subject. Accordingly, in an embodiment the disclosure includes a method of treating a cancer wherein the cancer cell is in a subject and the subject is administered an effective amount of a glycylcycline such as tigecycline. In an embodiment, the cancer cell is a hematological cancer cell. In another embodiment, the cancer cell is a solid cancer cell. In a further embodiment, the cancer cell is a cancer stem cell.

It is also demonstrated herein that tigecycline inhibits mammalian mitochondrial ribosome activity at a clinically achievable concentration. Accordingly, in an aspect, the disclosure includes a method of inhibiting a mammalian mitochondrial ribosome in a cell comprising contacting the cell with a glycylcycline such as tigecycline, for example for a suitable time and under suitable conditions, for example as under conditions described herein. In an embodiment, the method is for inhibiting a mammalian mitochondrial ribosome in a cell, in the absence of producing increased radical oxygen production.

Another aspect of the disclosure includes use of glycylcycline such as tigecycline for inhibiting a mammalian mitochondrial ribosome in a cell. In a further aspect, the disclosure includes use of a glycylcycline such as tigecycline for the manufacture of a medicament for inhibiting a mammalian mitochondrial ribosome in a cell. In an embodiment, inhibition of the mammalian mitochondrial ribosome is assessed by determining the level of a mitochondrial translated polypeptide. In an embodiment, the mitochondrial translated polypeptide is Cox-1. In another embodiment, the mitochondrial translated polypeptide is Cox-2. A person skilled in the art would recognize that any protein that is translated by mitochondrial ribosomes, preferably exclusively by mitochondrial ribosomes, can be assayed to assess inhibition of a mitochondrial ribosome. Without wishing to be bound to any particular theory, it is predicted that tigecycline, induces cell death by inhibiting mitochondrial ribosomal protein synthesis that thereby blocks oxidative phosphorylation and cellular metabolism and/or leads to disruption of the mitochondria.

In an embodiment, the glycylcycline comprises tigecycline. In another embodiment, the glycylcycline is tigecycline.

Cancers and cancer cells that can be treated include, but are not limited to, hematological cancers, including leukemia, lymphoma and myeloma, and solid cancers, including for example tumors of the brain (glioblastomas, medulloblastoma, astrocytoma, oligodendroglioma, ependymomas), lung, liver, thyroid, bone, adrenal, spleen, kidney, lymph node, small intestine, pancreas, colon, stomach, breast, endometrium, prostate, testicle, ovary, skin, head and neck, and esophagus.

In an embodiment, the cancer is a hematological cancer. In an embodiment, the hematological cancer is a leukemia. In another embodiment, the hematological cancer is a myeloma. In an embodiment, the hematological cancer is a lymphoma.

In an embodiment, the leukemia is selected from acute myeloid leukemia (AML), acute lymphocytic leukemia (ALL), chronic lymphocytic leukemia (CLL) and chronic myelogenous leukemia (CML). In an embodiment, the leukemia is AML. In an embodiment, the leukemia is ALL. In an embodiment, the leukemia is CLL. In a further embodiment, the leukemia is CML. In an embodiment, the cancer cell is a leukemic cell, for example, but not limited to, an AML cell, an ALL cell, a CLL cell or a CML cell.

In a further embodiment, the hematological cancer is a myeloma. In another embodiment, the hematological cancer cell is a myeloma cell.

In yet a further embodiment, the hematological cancer is a lymphoma. In an embodiment, the hematological cancer cell is a lymphoma cell.

In an embodiment, the cancer is a solid tumour cancer. In an embodiment, the solid tumour cancer is selected from ovarian cancer, prostate cancer and lung cancer. In an embodiment, the cancer cell is an ovarian cancer cell, a prostate cancer cell or a lung cancer cell.

In an embodiment, the glycylcycline, for example tigecycline, administered or contacted with the cell, is comprised in a composition, dosage or dosage form described herein.

In an embodiment, the composition comprises a glycylcycline such as tigecycline and, optionally, a suitable carrier or vehicle. In an embodiment, the composition comprises tigecycline and, optionally, a suitable carrier or vehicle. In an embodiment, the composition comprises an effective amount of a glycylcycline, for example tigecycline, and, optionally, a suitable carrier or vehicle.

In an embodiment, the composition is a pharmaceutical composition.

The compounds are suitably formulated into pharmaceutical compositions for administration to human subjects in a biologically compatible form suitable for administration in vivo.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Suitable vehicles are described, for example, in Remington's Pharmaceutical Sciences (2003-20^{th} edition). On this basis, the compositions include, albeit not exclusively, solutions of the substances in association with one or more than one pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which optionally further contain antioxidants, buffers, bacteriostats and solutes that render the compositions substantially compatible with the tissues or the blood of an intended recipient. Other components that are optionally present in such compositions include, for example, water, surfactants (such as Tween™), alcohols, polyols, glycerin and vegetable oils. Extemporaneous injection solutions and suspensions may be prepared from sterile powders, granules, tablets, or concentrated solutions or suspensions. The composition can be supplied, for example, but not by way of limitation, as a lyophilized powder which is reconstituted with sterile water or saline prior to administration to the subject.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound(s), together with a suitable amount of carrier so as to provide the form for direct administration to the subject.

In an embodiment, the compounds and compositions described herein are administered, for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

In an embodiment, the compound or composition is administered by intravenous infusion. In an embodiment, for example where the cancer is a solid tumour, the compound or composition is administered by direct intratumoral injection. In an embodiment, the compound or composition is administered by injection into tumour vasculature.

Wherein the route of administration is oral, the dosage form may be, for example, incorporated with excipient and used in the form of enteric coated tablets, caplets, gelcaps, capsules, ingestible tablets, buccal tablets, troches, elixirs, suspensions, syrups, wafers, and the like. The oral dosage form may be solid or liquid.

In an embodiment, the disclosure describes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. It is also possible to freeze-dry the compounds described herein and use the lyophilizates obtained, for example, for the preparation of products for injection.

In another embodiment, the disclosure describes a pharmaceutical composition wherein the dosage form is a liquid dosage form. A person skilled in the art would know how to prepare suitable formulations. Conventional procedures and ingredients for the selection and preparation of suitable formulations are described, for example, in Remington's Pharmaceutical Sciences (2003-20^{th} edition) and in The United States Pharmacopeia: The National Formulary (USP 24 NF19) published in 1999.

In another embodiment, the disclosure describes a pharmaceutical composition wherein the dosage form is an injectable dosage form. An injectable dosage form is to be understood to refer to liquid dosage forms suitable for, but not limited to, intravenous, subcutaneous, intramuscular, or intraperitoneal administration. Solutions of compounds described herein can be prepared in water suitably mixed with a surfactant such as hydroxypropylcellulose. Or for example, can be prepared in a sodium chloride solution, for example a 0.9% sodium chloride solution or a dextrose solution for example a 5% dextrose solution.

Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20^{th} edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In an embodiment, the dosage and/or each unit dosage form comprises from about 50 mg to about 1000 mg, from about 100 mg to about 2000 mg, from about 100 mg to about 1500 mg, from about 100 mg to about 1000 mg, from about 100 mg to about 700 mg, from about 100 mg to about 500 mg, from about 100 mg to about 350 mg, from about 100 mg to about 300 mg or from about 100 mg to about 250 mg of a glycylcycline, for example of tigecycline.

In another embodiment, the dosage and/or each unit dosage form comprises from about 150 mg to about 2000 mg, from about 150 mg to about 1500 mg, from about 150 mg to about 1000 mg, from about 150 mg to about 700 mg, from about 150 mg to about 500 mg, from about 150 mg to about 350 mg, from about 150 mg to about 300 mg or from about 150 mg to about 250 mg of a glycylcycline, for example of tigecycline.

In an embodiment, the dosage or dosage form comprises sufficient glycylcycline, for example tigecycline, to produce a peak serum concentration (i.e. Cₘₐₓ) from about 0.5 micrograms/mL to about 100 micrograms/mL, from about 0.5 micrograms/mL to about 80 micrograms/mL, from about 0.5 micrograms/ml to about 60 micrograms/mL, from about 0.5 micrograms/mL to about 40 micrograms/mL, from about 0.5 micrograms/mL to about 20 micrograms/mL, or from about 0.5 micrograms/mL to about 10 micrograms/mL.

In another embodiment, the dosage or dosage form comprises sufficient glycylcycline, for example tigecycline, to produce a peak serum concentration (i.e. Cₘₐₓ) from about 1 micrograms/mL to about 100 micrograms/mL, from about 10 micrograms/mL to about 100 micrograms/mL, from about 25 micrograms/ml to about 100 micrograms/mL, from about 40 micrograms/mL to about 100 micrograms/mL, from about 60 micrograms/mL to about 100 micrograms/mL, or from about 80 micrograms/mL to about 100 micrograms/m L.

As an example, a PK study was conducted in mice that received a single dose of tigecycline (50 mg/kg) i.p. The Cₘₐₓ was found to be 27+/4.5 μg/mL. The Tₘₐₓ was about 30 min and the half life was about 4.5 hours. Thus, the half life in mice is significantly shorter than the half life in humans (27 hours).

In an embodiment, the dosage form can alternatively comprise about 20 to about 100 mg of a glycylcycline/kg body weight, about 30 to about 100 mg of a glycylcycline/kg body weight, about 40 to about 100 mg of a glycylcycline/kg body weight, or about 50 to about 100 mg of a glycylcycline/kg body weight of a subject in need of such treatment formulated into a solid oral dosage form, a liquid oral dosage form, or an injectable dosage form. In another embodiment, the dosage form can comprise about 20 to about 90 mg of a glycylcycline/kg body weight, about 20 to about 80 mg of a glycylcycline/kg body weight, about 20 to about 70 mg of a glycylcycline/kg body weight, about 20 to about 60 mg of a glycylcycline/kg body weight, or about 20 to about 50 mg of a glycylcycline/kg body weight of a subject in need of such treatment formulated into a solid oral dosage form, a liquid oral dosage form, or an injectable dosage form.

It should be understood, that all of these dosages are exemplary, and any dosage in-between these points is also expected to be of use in the methods described herein.

In another aspect, the disclosure includes a method of identifying compounds, such as novel glycylcyclines, that are useful for example, for treating cancer, the method comprising: contacting a eukaryotic cell and/or cell extract comprising mitochondrial ribosomes with a test compound; and assessing whether mitochondrial ribosome function is decreased compared to a control, wherein a compound that decreases (e.g. inhibits) mitochondrial ribosomal function is a putative chemotherapeutic. A compound identified in such a screen is also useful for inhibiting mitochondrial ribosome function, for example, in research or other protocols. In an embodiment, the test compound is a glycylcycline. In an embodiment, the control is an untreated cell (e.g. a cell contacted with diluent). In a further embodiment, the control is a cell treated with tigecycline. In an embodiment, the compound is at least as inhibitory as tigecycline. In a further embodiment, mitochondrial ribosome function is assessed by determining a level of a mitochondrial ribosome translated polypeptide, preferably a polypeptide preferentially translated by, and more preferably, exclusively translated by, mitochondrial ribosomes. In a further embodiment, the mitochondrial ribosome translated polypeptide is Cox-1.

In another embodiment, the mitochondrial ribosome translated polypeptide is selected from ND1, ND2, ND3, ND4, ND4L, ND5, ND6, Cyt B, Cox-2, Cox-3, ATP6, and ATP8⁸ In another embodiment, mitochondrial ribosome function is assessed by determining the rate of oxidative phosphorylation and/or cellular metabolism wherein a decrease compared to a control is indicative that the compound is a putative chemotherapeutic. In an embodiment, the compound inhibitory at a concentration that is pharmacologically achievable and clinically relevant. A person skilled in the art would be familiar with methods for assessing mitochondrial ribosome function, including for example using western blot for assessing the level of a mitochondrial ribosome translated polypeptide ND1, ND2, ND3, ND4, ND4L, ND5, ND6, Cyt B, Cox-2, Cox-3, ATP6, and/or ATP8⁸.

### III. Kits

Another aspect of the disclosure is a kit for treating a cancer, inducing cytotoxicity in a cancer cell, or inhibiting a mammalian mitochondrial ribosome in a cell. In an embodiment, the kit comprises a glycylcycline such as tigecycline and instructions for use and/or packaging materials. In another embodiment, the kit comprises tigecycline and instructions for use and/or packaging materials.

The following non-limiting examples are illustrative of the present disclosure:

### EXAMPLES

### Example 1

Drug Repositioning as a Strategy to Rapidly Advance Novel Therapeutic Agents into Clinical Trial

Drug repositioning is a strategy to rapidly advance new therapeutic options into clinical trial and has been shown to have clinical efficacy. The repositioning of thalidomide as a therapeutic agent for the treatment of myeloma and myelodysplasia is one of the best-known examples of this strategy, but there have been multiple other successes. For example, the broad spectrum antiviral ribavirin was found to suppress oncogenic transformation by disrupting the function and subcellular localization of the eukaryotic translation initiation factor eIF4E^{9,10}. As such, ribavirin was recently evaluated in a phase I dose escalation study in patients with relapsed or refractory M4/M5 acute myeloid leukemia (AML). In this study of 13 patients treated with ribavirin, there was 1 complete remission, and 2 partial remissions. Thus, ribavirin may be efficacious for the treatment of AML¹¹. Likewise, the anti-fungal ketoconazole inhibits the production of androgens from the testes and adrenals in rats. Given this finding, ketoconazole was rapidly advanced into clinical trials for patients with prostate cancer where it displayed clinical efficacy in early studies^{12,13}.

### Tigecycline

To identify compounds active against leukemia stem cells, a library of drugs (n=312) with well-characterized pharmacokinetics and toxicology and a wide therapeutic window was compiled. This library was then screened to identify agents that reduced the viability of TEX and M9-ENL1 cells. TEX and M9-ENL1 cells were derived from lineage-depleted human cord blood cells (Lin-CB) transduced with TLS-ERG or MLL-ENL oncogenes, respectively, and, as shown previously, display properties of stem cells including hierarchal differentiation and marrow repopulation^{14,16}. In this screen, TEX and M9-ENL1 cells were treated with aliquots of the compounds. After incubation, cell growth and viability was measured by the MTS assay. From this screen, tigecycline was identified.

Tigecycline is a anti-microbial agent of the glycylcycline class that is active against a range of gram-positive and gram-negative bacteria, particularly drug-resistant pathogens¹⁶ and FDA-approved for the treatment of complicated gram positive and gram negative infections. Tigecycline was developed synthetically as an analogue to minocycline with the addition of a tert-butyl-glycylamido side chain to the tetracycline backbone¹⁷. This approach was used to decrease drug resistance effects mediated by efflux pumps and improve its affinity for the ribosome. Consistent with its design, tigecycline has been shown to inhibit bacterial protein synthesis 3- and 20-fold greater than minocycline and tetracycline respectivelyl⁸. Mechanistically, tigecycline reversibly binds to the 30S subunit of the bacterial ribosome, blocking the aminoacyl-tRNA form entering the A site¹⁹, thereby inhibiting elongation of the peptide chain and protein synthesis.

Tigecycline is routinely administered as 50 mg intravenously every 12 hours without significant toxicity, but higher doses have also been used safely. For example, intravenous doses of 300 mg are well tolerated save for mild nausea and produce a Cmax of 2.82 μg/mL (5 μM)²⁰, a concentration within the range required for anti-leukemic effects. Toxicology studies in animals have been conducted. Rats receiving >30 mg/kg/day×2 weeks developed reversible anemia, thrombocytopenia, and leucopenia with a hypocellular bone marrow²¹. The dose of 30 mg/kg translates to 150 mg of drug in humans based on scaling for body surface area and weight, and is within 3 times the antimicrobial dose of drug. However, these higher concentrations of tigecycline are not used in the treatment of infection, potentially explaining why anti-cancer activity has not been previously reported with the drug. Further, animal studies have demonstrated that the drug accumulates in tissues such as the bone and bone marrow with ratios to the plasma as high as 19:1.

### Mitochondrial Protein Synthesis

Mechanistic studies described herein demonstrate that tigecycline inhibits mitochondrial protein synthesis. Eukaryotic cells have two separate genomes; nuclear DNA organized in chromosomes, and the circular mitochondrial DNA located within mitochondria. Mitochondrial DNA is comprised of double-stranded circular genome 16.6 kbp in length, and lacking introns²². It encodes two rRNAs, 22 t-RNAs and 13 of the 90 proteins in the mitochondrial respiratory chain. The remaining proteins of the respiratory chain are nuclear-encoded, imported into the mitochondria and assembled into the functional complexes of electron transport chain.

Mitochondrial ribosomes differ from bacterial and eukaryotic cytosolic ribosomes in their structure, and chemical properties²³. Compared to bacterial ribosomes, mitochondrial ribosomes have approximately half as much rRNA and over twice the amount of protein. Mitochondrial ribosomal proteins are encoded by nuclear genes and translated in the cytosol. Once translated, these proteins are imported into the mitochondria where they join two rRNA molecules to form the functional ribosomes of the mitochondria. Many of these mitochondrial ribosomal proteins have no similar analogues in bacterial or cytosolic ribosomes. Although mitochondrial ribosomes differ structurally from cytoplasmic and bacterial ribosomes, they function similarly. In addition, mitochondrial and cytoplasmic ribosomes use the same elongation initiation machinery²⁴⁻²⁶.

Antibiotics that inhibit bacterial protein synthesis have been reported to cross-react with human mitochondrial ribosomes and inhibit mitochondrial protein synthesis²⁷. For example, chloramphenicol can cause bone marrow suppression, which has been attributed to inhibition of mitochondrial protein synthesis inhibition by binding the A site of the mitochondrial ribosome²⁸. Oxazolidinones, which bind to the same bacterial ribosome site as chloramphenicol, also can cause myelosuppression and inhibit human mitochondrial ribosomes²⁹.

### Methods

### Reagents

The compounds in the chemical library were purchased from Sequoia Research Products Limited (Pangbourne, United Kingdom). Annexin V, and Propidium Iodide (PI), were purchased from (Invitrogen Canada, Burlington, Canada).

### Cell Lines

Human leukemia (OCI-AML2, HL60, U937) cell lines were maintained in RPMI 1640 medium. Myeloma (LP-1, KMS11, 8226, JJN3, OP-M2) cell lines were maintained in Iscove's media. Ovarian (OVCAR), prostate (PC3), and lung alveolar (A549) cell lines were maintained in RPMI 1640 medium. Media was supplemented with 10% fetal calf serum (FCS), 100 μg/mL penicillin and 100 units/mL of streptomycin (all from Hyclone, Logan, Utah). TEX cells were maintained in IMDM, 15% FBS, 2 mM L-glutamine, 1%, penicillin-streptomycin, 20 ng/mL SCF, 2 ng/mL IL-3. M9-ENL1 cells were maintained in alpha-MEM, 20% FBS, 5% human plasma, 2 mM L-glutamine, 1%, penicillin-streptomycin, 100 ng/mL SCF, 10 ng/mL IL-3, 5 ng/mL IL-7, and 5 ng/mL FLT3L. All cells were incubated at 37° C. in a humidified air atmosphere supplemented with 5% CO₂.

### Primary Cells

Primary human acute myeloid leukemia (AML) samples were isolated from fresh peripheral blood samples of consenting patients with AML. Similarly, primary normal hematopoietic cells were obtained from healthy consenting volunteers donating peripheral blood mononuclear cells (PBSC) for stem cell transplantation. The mononuclear cells were isolated from the samples by Ficoll density centrifugation. Primary cells were cultured at 37° C. in IMDM supplemented with 20% FCS, 1 mM of L-glutamine and appropriate antibiotics. The collection and use of human tissue for this study were approved by the University Health Network institutional review board.

### Chemical Screen

TEX, and M9-ENL-1 cells were seeded into 96-well polystyrene tissue culture plates (Corning). After seeding, cells were treated with 5 μL aliquots of the chemical library (n=312) at final concentrations of 10 μM and 1 μM (DMSO 0.025%). 72 (TEX) and 48 (M9-ENL-1) hours after incubation, cell growth and viability was measured by MTS assay. Liquid handling was performed by a Biomek FX Laboratory Automated Workstation (Beckman Coulter Fullerton, Calif.).

### Cell Viability Assays

Cell growth and viability was assessed by the MTS assay (Promega, Madison, Wis.) according to the manufacturer's instructions. Apoptosis and cell death was measured by Annexin V-fluoroscein isothiocyanate (FITC; Biovision Research Products, Mountain View, Calif.), propidium iodide staining and flow cytometry according to the manufacturer's instructions and as previously described³⁰.

To assess clonogenic growth, primary AML cells (1.0×10⁵/mL) or granulocyte colony-stimulating factor (G-CSF) mobilized PBSCs (1.0×10⁵/mL) were plated in duplicate with increasing concentrations of tigecycline in MethoCult GF H4434 medium (StemCell Technologies, Vancouver, BC) containing 1% methycellulose in IMDM, 30% FCS, 1% bovine serum albumin, 3 U/mL of recombinant human erythropoietin, 10⁻⁴ M of 2-mercaptoethanol, 2 mM of L-glutamine, 50 ng/mL of recombinant human stem cell factor, 10 ng/mL of GM-CSF, and 10 ng/mL of rh IL-3). Seven days (AML samples) or 14 days (normal PBCS) after plating, the number of colonies was counted as previously described³¹.

### Assessment of Tigecycline's Anti-Leukemia Activity in Mouse Models of Leukemia

OCI-AML2 human leukemia cells (1×10⁶) were injected subcutaneously into the flanks of SCID mice (Ontario Cancer Institute, Toronto, ON). Seven days after injection, once tumours were palpable, mice were treated with tigecycline twice daily (50 mg/kg or 100 mg/kg by i.p. injection) or vehicle control (n=10 per group) for 14 days. Tumor volume (tumor length×width²×0.5236) was measured three times a week using calipers. Twenty-one days after injection of cells, mice were sacrificed, tumors excised and the volume and mass of the tumors were measured.

To assess tigecycline in mouse models of primary AML, primary human AML cells were isolated form a fresh peripheral blood sample from a patient with AML. A frozen aliquot was thawed, counted and resuspended in PBS. Primary cells (2×10⁶) were injected into the right femur of 10 week old female NOD-SCID mice that were irradiated 24 hours previously with 208 rad from a cesium-137 source. Three weeks after injection of the AML cells, mice were treated with tigecycline (100 mg/kg by i.p. injection) daily or vehicle control (n=10 per group) for three weeks. Mice were then sacrificed and the cells were flushed from the femurs. Engraftment of human AML cells into the marrow was assessed by enumerating the percentage of human CD45⁺CD33⁺CD19⁻ by flow cytometry.

All animal studies were carried out according to the regulations of the Canadian Council on Animal Care and with the approval of the local ethics review board.

### Immunoblotting

Total cell lysates were prepared from cells as described previously³². Briefly, cells were washed with phosphate buffered saline pH 7.4 twice and suspended in lysis buffer (1.5% n-dodecyl β-maltoside, Sigma Aldrich, St. Louis, Mo.) containing protease inhibitor tablets (Complete tablets; Roche, Ind.). Protein concentrations were measured by the DC Protein assay. Equal amounts of protein were subjected to sodium dodecyl sulphate (SDS)-polyacrylamide gels followed by transfer to nitrocellulose membranes. Membranes were probed with anti-Cox-1 (Santa Cruz Biotechnology Inc), anti-grp78 (Sigma Aldrich, St. Louis, Mo.), anti-XIAP (BD Biosciences), anti-α-tubulin (Sigma Aldrich, St. Louis, Mo.), anti-β-actin (Cell signaling Technology), and secondary antibodies from GE Health (IgG peroxidase linked species-specific whole antibody). Detection was performed by the enhanced chemical luminescene method (Pierce, Rockford, Ill.).

### Detection of Mitochondrial Membrane Potential

To measure mitochondrial membrane potential, cells were treated with tigecycline similarly as described above and then washed twice with PBS and incubated with 5 μM of 5,5′,6,6′-tetrachloro-1,1′,3,3′-tetraethyl benzimidazolylcarbocyanine iodide (JC-1, Sigma-Aldrich) for 20 minutes at 37° C. Each sample was then washed twice with 1 mL PBS and resuspended in 500 μL PBS prior to being read on a BD FACSCalibur. Samples were excited at 488 nm and emission was collected at 526 nm (green) and 595 nm (red). Analysis was conducted using FlowJo software (TreeStar Inc). To obtain the mitochondrial membrane potential (red/green), emission from the red channel was divided by emission from the green channel.

### Reverse-Transcriptase Real-Time PCR

First-strand cDNA was synthesized from 1 μg of DNase-treated total cellular RNA using random primers and SuperScript II reverse transcriptase (Invitrogen) according to the manufacturer's protocols. Real-time PCR assays were performed in triplicate with 5 ng of RNA equivalent cDNA, SYBR Green PCR Master mix (Applied Biosystems), and 400 nmol/L of gene-specific primers. Reactions were processed and analyzed on an ABI 7900 Sequence Detection System (Applied Biosystems). Forward/reverse PCR primer pairs for human cDNAs for human Cox-1 and 18S were used. Relative mRNA expression was determined using the CT method as described³².

### Results

Chemical Screen Identifies Tigecycline with Potential Anti-Leukemia Activity

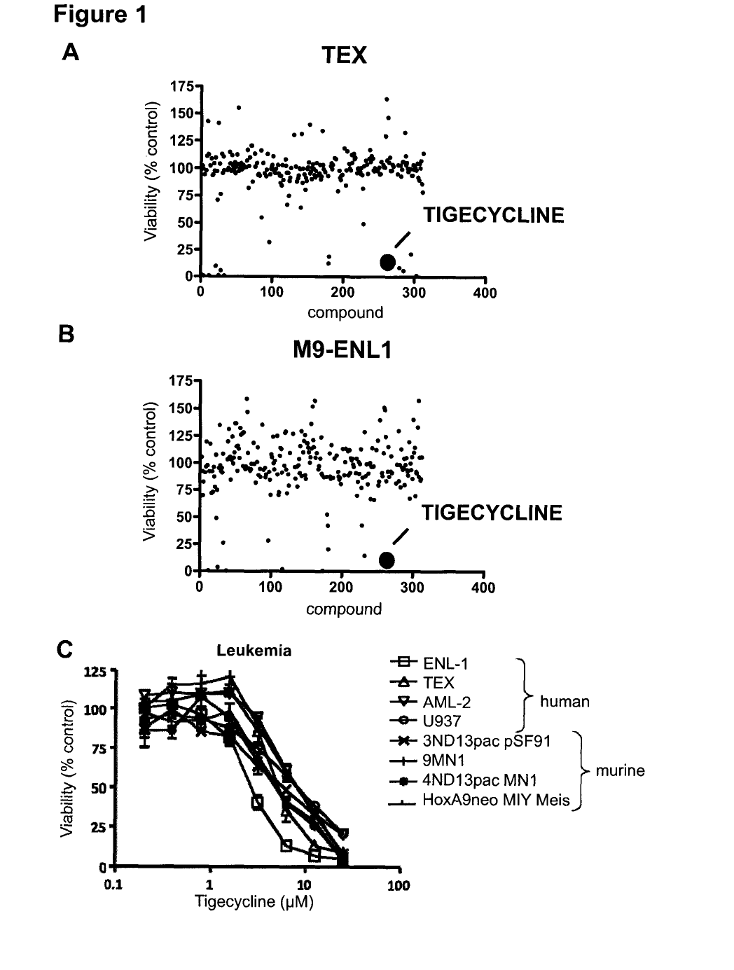

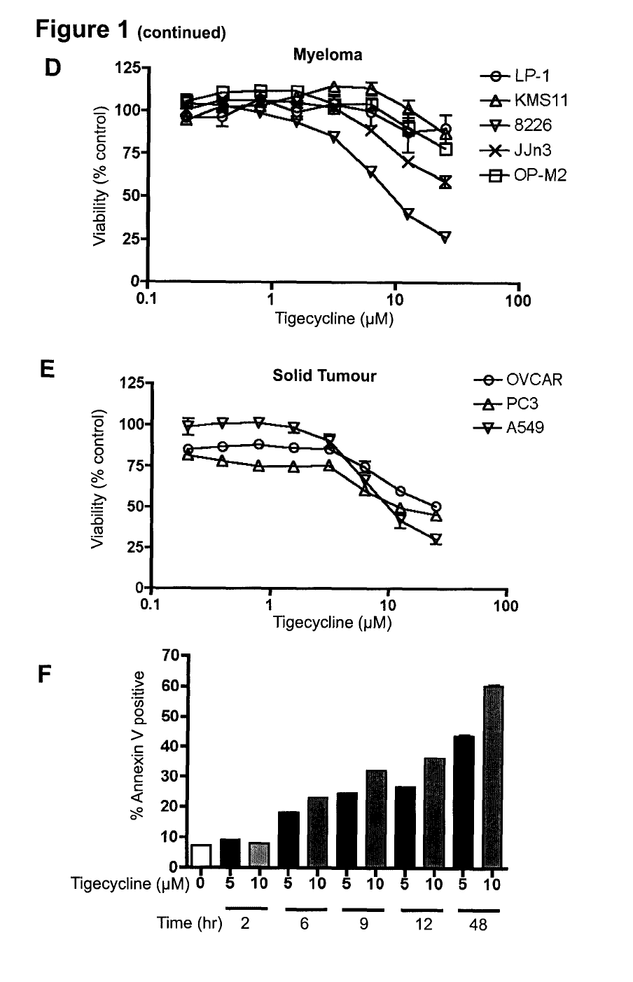

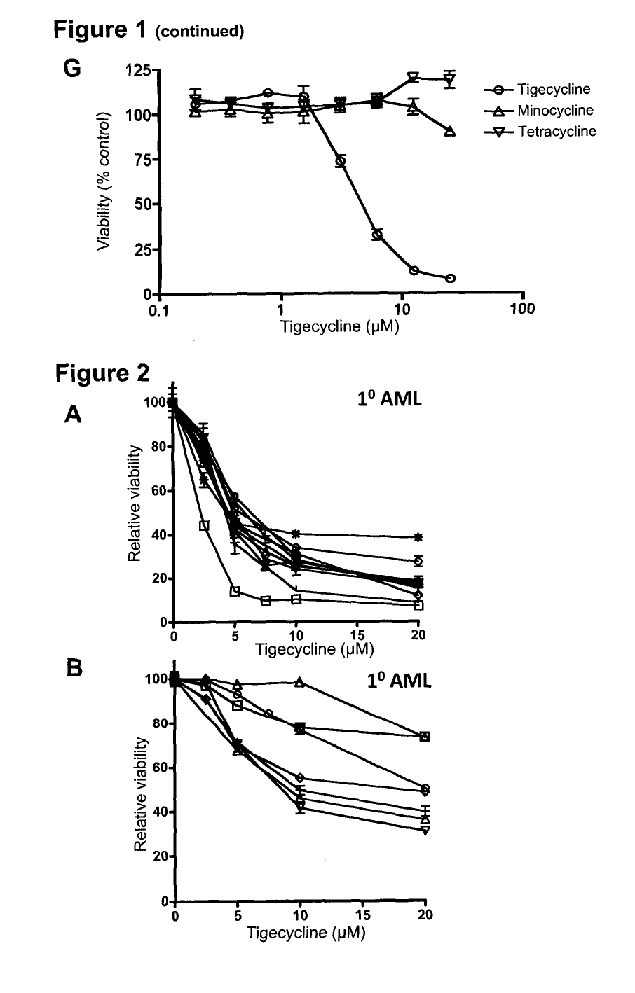

FDA-approved drugs with previously unrecognized against leukemia and leukemia stem cells can be rapidly repositioned for this new indication given their prior toxicology and pharmacology testing. To identify such compounds, a chemical library (n=312) of drugs with wide therapeutic windows and well-understood pharmacokinetics was compiled. TEX and M9-ENL1 leukemia cells were treated with aliquots of this library at concentrations of 10 μM and 1 μM. After incubation (TEX 72 hours, M9-ENL1 48 hours), cell growth and viability was measured by the MTS assay. Differences in times of incubation were due to differences in growth rates between the two lines. From these screens, tigecycline was identified as cell growth in both TEX and M9-ENL1 cells at 10 μM. The results of this screen at drug concentrations of 10 μM are shown in FIGS. 1A, B.

### Tigecycline has Preferential Anti-Leukemic Activity In Vitro

To assess the effects of tigecycline on the growth and viability of malignant cell lines, a panel of leukemia, myeloma and solid tumour cells were treated with increasing concentrations of tigecycline. Seventy-two hours after incubation, cell growth and viability was assessed by the MTS assay. Tigecycline decreased the viability of the tested leukemia cell lines with an IC50 of 5 to 8 μM (FIG. 1C). The murine leukemia cell lines are derived from mouse bone marrow with various inducers of pre-leukemic and leukemic phenotypes. 3ND13pac pSF91 cells are representative of a pre-leukemic model, which can be induced to AML with secondary hits (Meis1, MN1). 9MN1 cells are transduced with the oncogene meningioma 1 (MN1) (34; PMID: 17494859) and are capable of aggressive AML induction in mouse models. ND13pan MN1 cells are engineered to express both MN1 and ND13 oncogenes. Both 9MN1 and ND13pan MN1 cells maintain high frequencies of leukemic stem cells. HoxA9neo Meis1 cells co-express HOXA9 and Meis1 oncogenes, and are capable of transplantable AML induction in mouse models. In contrast, tigecycline was less cytotoxic to myeloma and solid tumour cells lines with IC50 over 10 μM (FIGS. 1D, E). Tigecycline displays time-dependent increases in apoptosis in TEX cells, determined by flow cytometry as percentage of cells labeled by Annexin V (FIG. 1F). Of note, although tigecycline is a structural analogue of minocycline and tetracycline, TEX cells were not sensitive to either minocycline or tetracycline at concentrations up to 25 μM (FIG. 1G).

### Tigecycline Induces Cell Death in Primary AML Cells Preferentially Over Normal Hematopoietic Cells

Given the cytotoxicity of tigecycline towards leukemia cell lines, the ability of tigecycline to induce cell death in primary acute myeloid leukemia (AML) patient samples and normal hematopoietic cells was compared and evaluated. Primary AML patient samples and primary normal hematopoietic cells were treated for 48 hours with increasing concentrations of tigecycline. After incubation, cell viability was measured by Annexin V staining.

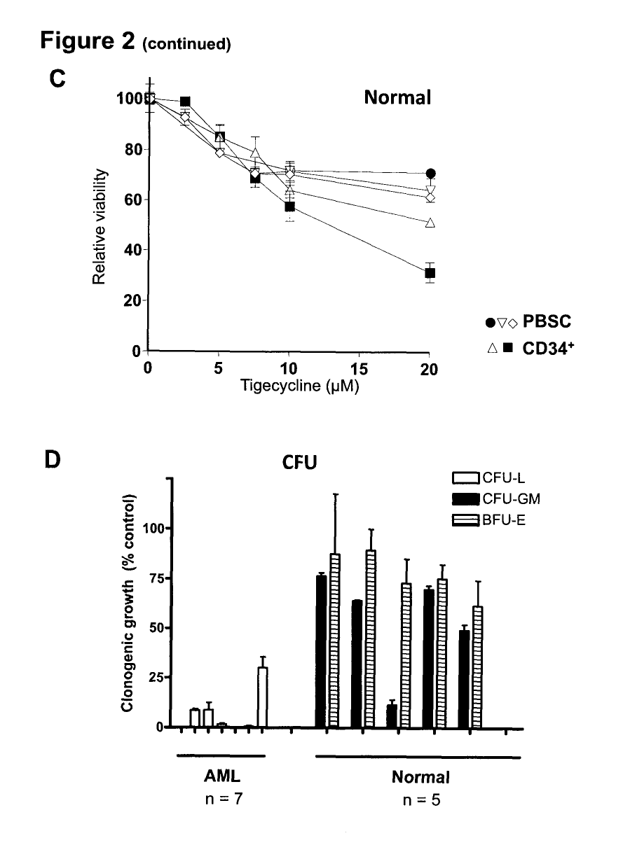

A subset of leukemia patients, displayed sensitivity to tigecycline (LD₅₀ 5 μM, n=13 FIG. 2A), while a smaller group of patients were more resistant to tigecycline treatment in vitro (LD₅₀>9 μM, n=7, FIG. 2B). Two of the sensitive AML patient samples were refractory to all current standard AML chemotherapy regimens. Primary normal hematopoietic cells (PBSC) were extracted from the peripheral blood of consenting donors who had been G-CSF mobilized for allogeneic bone marrow transplantation. These normal hematopoietic cells were more resistant to tigecycline than sensitive primary AML samples (LD₅₀>10 μM, n=5, FIG. 2C). When the CD34⁺ progenitor fraction of these normal hematopoietic cells was analyzed for tigecycline sensitivity, similar activity was seen compared to PBSC. Tigecycline's ability to inhibit the clonogenic growth of primary AML and normal hematopoietic cells in methylcellulose colony formation assays was assessed. Tigecycline (5 μM) reduced the clonogenic growth of primary AML patient samples (n=7) by 93±4% (FIG. 2D). In contrast, 5 μM tigecycline reduced the clonogenic growth of normal hematopoietic cells by 34±5% (n=5) (FIG. 2D). Thus, tigecycline induced cell death and inhibited the clonogenic growth of AML cell lines and primary patient samples preferentially over normal cells at pharmacologically achievable concentrations.

### Tigecycline Demonstrates Activity in Mouse Models of Leukemia

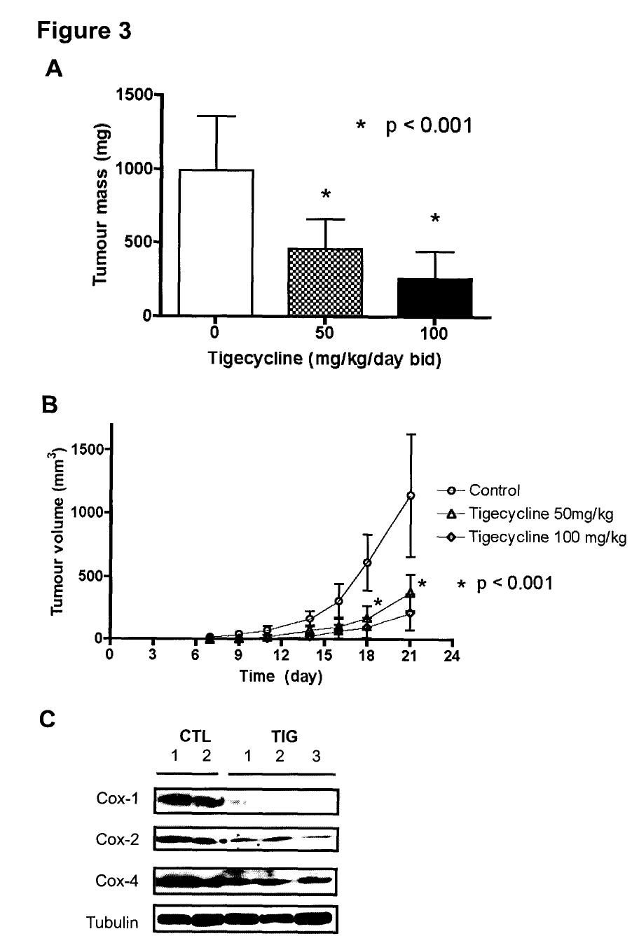

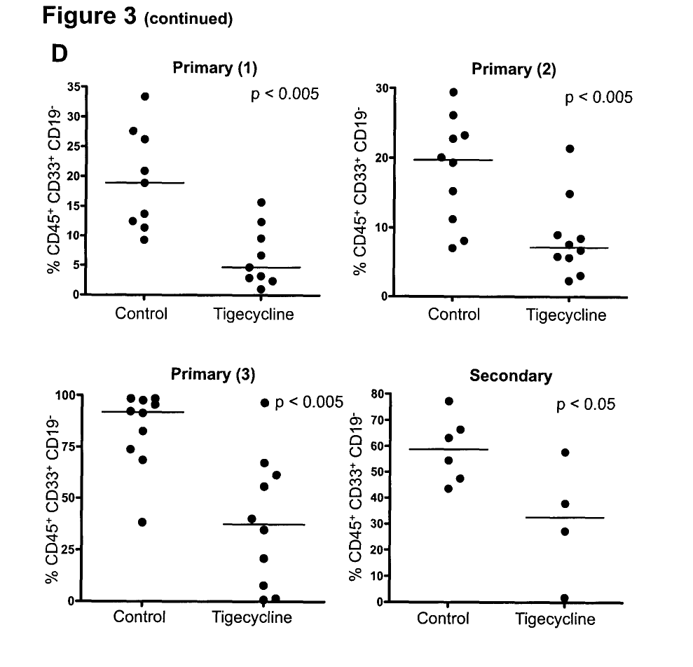

Given the effects of tigecycline as a potential anti-leukemia agent, tigecycline in mouse models of leukemia was evaluated. To evaluate the anti-tumor efficacy of tigecycline in vivo, human OCI-AML2 leukemia cells were injected into the flank of SCID mice. Seven days after injection, once tumours were palpable, mice were treated with tigecycline twice daily (50 mg/kg or 100 mg/kg by i.p. injection) or vehicle control (n=10 per group) for 14 days. Tumor volume and mass were measured over time (FIGS. 3A, B). Compared to vehicle control, tigecycline significantly (p<0.001) decreased tumor mass and volume by up to 70%. No evidence of toxicity from tigecycline was observed. Specifically, there was no change in body or behavior of the mice. At the conclusion of the experiment, there were no gross changes to the organs at necropsy. To determine whether in vivo tigecycline treatment was inhibiting mitochondrial translation in the tumour cells, the relative expression of cytochrome C oxidase protein subunits isolated from tumours of tigecycline-treated mice (50 mg/kg bid for 5 days) and vehicle-treated mice (saline bid for 5 days) were examined. Tumours from tigecycline-treated mice demonstrate a preferential decreased expression of mitochondrially translated subunits Cox-1 and Cox-2 relative to nuclear-translated and encoded subunit Cox-4. Therefore, the decreased tumour mass and volume in the OCI-AML2 xenograft model was associated with mitochondrial translation inhibition (FIG. 3C).

The effects of tigecycline on primary AML stem cells defined by their ability to initiate leukemic engraftment in vivo were also assessed. Primary cells from three patients (three separate experiments) with AML were injected intra-femorally into the right femur of female sublethally irridated NOD-SCID mice. Three weeks after injection, mice were treated with tigecycline (100 mg/kg by i.p. injection) daily or vehicle control (n=10 per group) for three weeks. Six weeks after injection, mice were sacrificed and the right femur flushed of cells. Engraftment of human AML cells in the marrow was assessed by enumerating the percentage of human CD45⁺CD33⁺CD19⁻ cells by flow cytometry. Compared to mice treated with vehicle control, tigecycline significantly decreased the engraftment of human AML primary cells without gross organ toxicity or loss of body weight (FIG. 3D).

Thus, tigecycline delays growth of leukemia tumors and decreases engraftment of primary AML cells in mouse models of leukemia at pharmacologically achievable concentrations.

### Tigecycline Inhibits Mitochondrial Protein Synthesis in Leukemia Cells

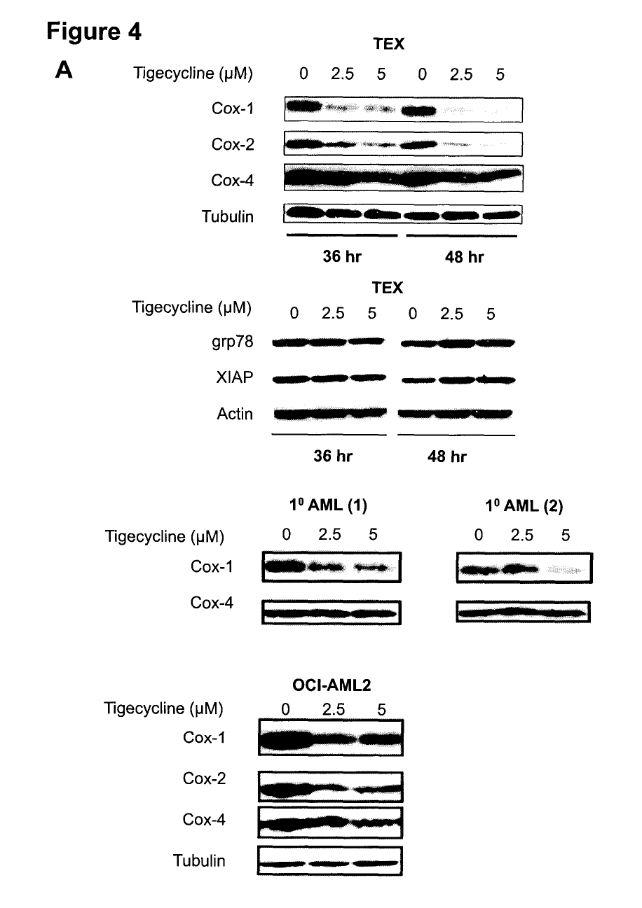

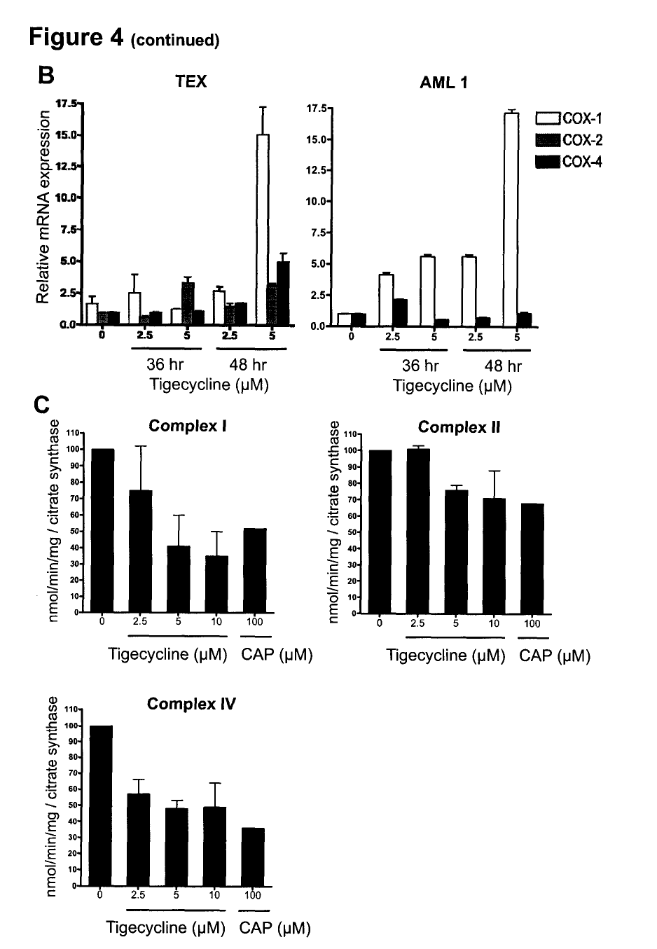

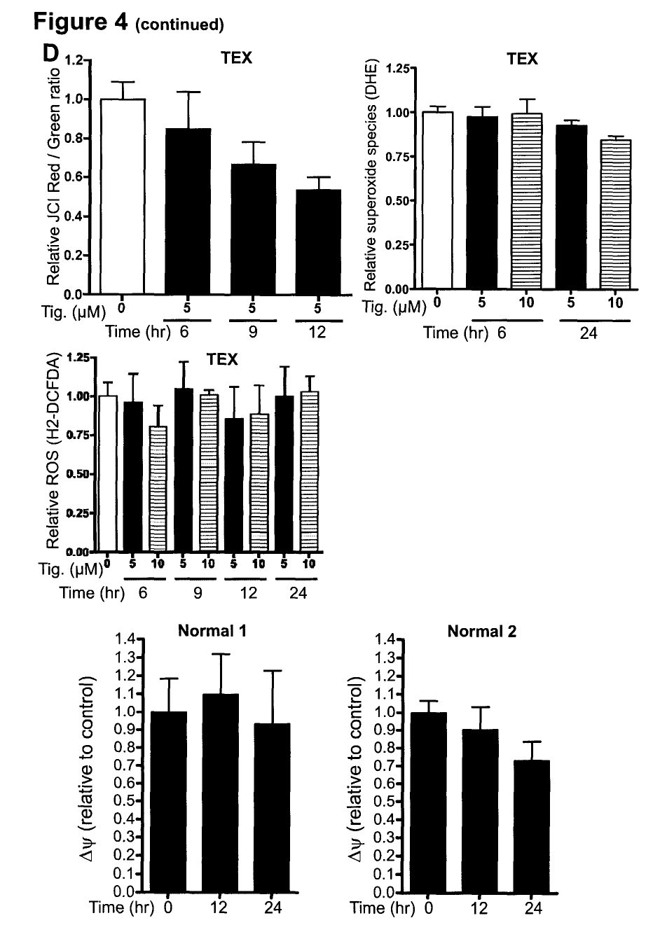

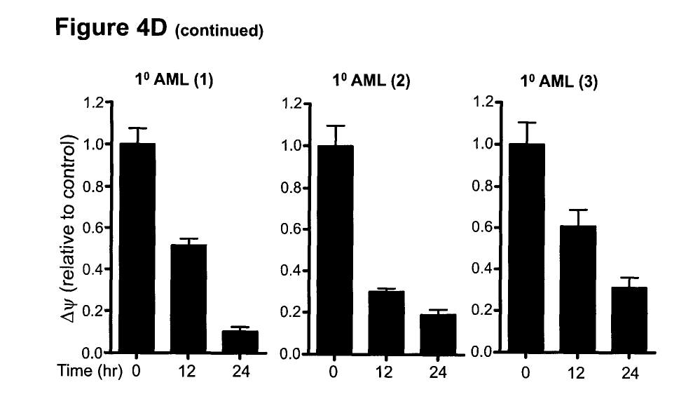

Tigecycline binds to the 30S bacterial ribosome, thereby inhibiting elongation, and decreasing protein synthesis. Therefore, the effects of tigecycline on the expression of proteins whose translation was dependent on cytosolic and mitochondrial ribosomes was evaluated. Cells from TEX, OCI-AML2 and two primary AML patients were treated with increasing concentrations of tigecycline and levels of the mitochondrially translated proteins Cox-1, Cox-2 and Cox-4 were measured over time by immunoblotting. Cox-1 and Cox-2 are two of the 3 large subunits (I, II and III) of cytochrome C oxidase, the terminal enzyme of the electron transport chain in mitochondria. The subunits I, II and III are encoded by the mitochondrial genome, and are exclusively translated by mitochondrial ribosomes³³. Contrary to Cox-1 and Cox-2, the Cox-4 subunit of cytochrome C oxidase is encoded by the nuclear genome, and translated by nuclear ribosomes. TEX, OCI-AML2 and cells from two AML patients were treated with increasing concentrations, and the expression of Cox subunit proteins was examined by Western blotting. Tigecycline treatment is associated with preferential decrease of mitochondrially-translated subunits I and II, over nuclear-translated subunit IV (FIG. 4A). Also, tigecycline did not alter the expression of the nuclear-encoded proteins XIAP or grp78, which are translated by cytosolic ribosomes (FIG. 4A). TEX and primary AML cells treated with tigecycline had preferential increased mRNA expression of mitochondrial-encoded genes Cox-1, Cox-2, over the nuclear-encoded gene Cox-4 (FIG. 4B) as determined by quantitative RT-PCR. This is consistent with previous studies which have shown that mitochondrial protein synthesis inhibition results in an increase in mitochondrially encoded mRNA, and stabilized, unchanged nuclear-encoded mRNA of mitochondrial enzyme subunits.

The effect of tigecycline treatment on enzyme activity of the respiratory chain complexes relative to the mitochondrial non-respiratory chain enzyme citrate synthase was examined. TEX leukemia cells were treated with tigecycline for seventy-hours and then respiratory chain enzyme relative to citrate synthase activity was analyzed. Equivalent tigecycline concentrations (2.5 μM and 5 μM) decreased enzyme activities of Complexes I and IV relative to citrate synthase, while complex II activity was less affected (FIG. 4C). Similar results were seen with chloramphenicol, a known inhibitor of mitochondrial protein synthesis.

During oxidative phosphorylation, the intermediate between oxygen reduction and ATP synthesis is the electrochemical proton gradient, comprised mostly of the mitochondrial membrane potential. ATP synthase (complex V) uses the proton gradient as an electromotive force to generate ATP. The effect of tigecycline treatment on the mitochondrial membrane potential as determined by the carbocyanine dye JC-1 was examined. TEX cells and three different primary AML samples had decreased mitochondrial membrane potential after tigecycline treatment (5 μM) at times preceding onset of cell death (FIG. 4D). Loss of mitochondrial membrane potential was not seen on normal hematopoietic cells from two different G-CSF mobilized normal donors. AML samples also had increased mitochondrial DNA copy number and increased mitochondrial mass as described in Example 2 (FIG. 5). This is most likely associated with the preferential sensitivity of primary leukemic blast cells towards tigecycline compared to normal hematopoietic cells.

A byproduct of the mitochondrial enzyme activities of the electron transport chain is the generation of reactive oxygen species (ROS). Inhibitors of the mitochondrial complexes have been previously shown to produce rapid increases in ROS generation. Therefore, the role of tigecycline on ROS generation in leukemia cells by analyzing hydrogen peroxide ions (H₂O₂) using a dichlorofluorescein dye (DCF-DA), and superoxide anions using dihydroethidium (DHE) was examined. There was no observed increase in ROS generation in TEX cells after treatment with tigecycline (FIG. 4D) at time-points up to 24 hours. This unchanged ROS generation with tigecycline treatment was unique when compared to the activity of various mitochondrial complex enzyme inhibitors, such as sodium azide, rotenone, oligomycin or antimycin, where rapid increases in ROS levels were observed. Tigecycline treatment in leukemia cells is resulting in mitochondrial membrane potential dissipation via different mechanisms than those routinely seen with mitochondrial ETC inhibitors or uncoupling agents (FIG. 4D).

### Example 2

The mitochondrial characteristics of acute myeloid leukemia cells were assessed.

Mitochondrial DNA copy number was determined in mononuclear cells from the peripheral blood of primary AML and normal G-CSF mobilized donors. DNA was extracted from cells and real-time PCR was performed for mitochondrial ND1 relative to human globulin (HGB). ND1/HGB ratio is shown relative to cells from one normal G-CSF mobilized donor (FIG. 5A).

Mitochondrial mass was also assessed. Mitochondrial mass was assessed in AML bulk blasts and CD45+/CD34+ cells and compared to CD45+/CD34+ cells from normal G-CSF mobilized individuals. Mitochondrial mass was measured by incubating cells with Mitotracker Green FM dye, and subsequent flow cytometry.

Briefly AML patient samples were treated with 5 and 10 uM of tigecycline for 48 hours. After treatment, cell viability was measured by Annexin V staining. In parallel, the same AML cells not treated with tigecycline were stained with Mitotracker Green FM to measure mitochondrial mass. Mitochondrial mass was normalized to a sample of normal CD34+ hematopoietic cells.

Median fluorescence intensity is shown relative to one normal G-CSF mobilized donor (FIG. 5B).

Mitochondrial mass was measured in blasts from eleven AML patients using the previously described Mitotracker Green FM method. Cells were then incubated with increasing concentrations of tigecycline for 48 hours. Viability was assessed by Annexin-V/PI staining and subsequent flow cytometry. The correlation between baseline mitochondrial mass and sensitivity to tigecycline at doses of 5 μM and 10 μM is shown (FIG. 5C).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. TILL J E, McCULLOCH E A. A direct measurement of the radiation sensitivity of normal mouse bone marrow cells. Radiat Res. Vol. 14; 1961:213-222.
2. Lapidot T, Sirard C, Vormoor J, et al. A cell initiating human acute myeloid leukaemia after transplantation into SCID mice. Nature. Vol. 367; 1994:645-648.
3. Guzman M L, Neering S J, Upchurch D, et al. Nuclear factor-kappaB is constitutively activated in primitive human acute myelogenous leukemia cells. Blood. Vol. 98; 2001:2301-2307.
4. Dean M, Fojo T, Bates S. Tumour stem cells and drug resistance. Nat Rev Cancer. Vol. 5; 2005:275-284.
5. Konopleva M, Zhao S, Hu W, et al. The anti-apoptotic genes Bcl-X(L) and Bcl-2 are over-expressed and contribute to chemoresistance of non-proliferating leukaemic CD34+ cells. Br J. Haematol. Vol. 118; 2002:521-534.
6. Sum P E. Case studies in current drug development: ‘glycylcyclines’. Curr Opin Chem. Biol. 2006; 10:374-379.
7. Sum P E, Lee V J, Testa R T, et al. Glycylcyclines. 1. A new generation of potent antibacterial agents through modification of 9-aminotetracyclines. J Med. Chem. 1994; 37:184-188.
8. Ott M, Herrmann J M. Co-translational membrane insertion of mitochondrially encoded proteins. Biochim Biophys Acta; 2009.
9. Kentsis A, Topisirovic I, Culjkovic B, Shao L, Borden K L. Ribavirin suppresses eIF4E-mediated oncogenic transformation by physical mimicry of the 7-methyl guanosine mRNA cap. Proc Natl Acad Sci USA. 2004; 101:18105-18110.
10. Tan K, Culjkovic B, Amri A, Borden K L. Ribavirin targets eIF4E dependent Akt survival signaling. Biochem Biophys Res Commun. 2008; 375:341-345.
11. Assouline S, Culjkovic B, Cocolakis E, et al. Molecular targeting of the oncogene eIF4E in acute myeloid leukemia (AML): a proof-of-principle clinical trial with ribavirin. Blood. 2009; 114:257-260.
12. Sella A, Kilbourn R, Amato R, et al. Phase II study of ketoconazole combined with weekly doxorubicin in patients with androgen-independent prostate cancer. J Clin Oncol. 1994; 12:683-688.
13. Small E J, Halabi S, Dawson N A, et al. Antiandrogen withdrawal alone or in combination with ketoconazole in androgen-independent prostate cancer patients: a phase III trial (CALGB 9583). J Clin Oncol. 2004; 22:1025-1033.
14. Warner J K, Wang J C Y, Takenaka K, et al. Direct evidence for cooperating genetic events in the leukemic transformation of normal human hematopoietic cells. Leukemia. Vol. 19; 2005:1794-1805.
15. Barabé F, Kennedy J A, Hope K J, Dick J E. Modeling the initiation and progression of human acute leukemia in mice. Science. Vol. 316; 2007:600-604.
16. Stein G E, Craig W A. Tigecycline: a critical analysis. Clin Infect Dis. Vol. 43; 2006:518-524.
17. Garrison M W, Neumiller J J, Setter S M. Tigecycline: an investigational glycylcycline antimicrobial with activity against resistant gram-positive organisms. Clinical therapeutics. Vol. 27; 2005:12-22.
18. Olson M W, Ruzin A, Feyfant E, Rush T S, O'Connell J, Bradford P A. Functional, biophysical, and structural bases for antibacterial activity of tigecycline. Antimicrob Agents Chemother. Vol. 50; 2006:2156-2166.
19. Doan T-L, Fung H B, Mehta D, Riska P F. Tigecycline: a glycylcycline antimicrobial agent. Clinical therapeutics. Vol. 28; 2006:1079-1106.
20. Muralidharan G, Micalizzi M, Speth J, Raible D, Troy S. Pharmacokinetics of tigecycline after single and multiple doses in healthy subjects. Antimicrob Agents Chemother. Vol. 49; 2005:220-229.
21. Wyeth-Canada. Tygacil: Tigecycline for injection. Product Monograph; 2007.
22. Lang B F, Gray M W, Burger G. Mitochondrial genome evolution and the origin of eukaryotes. Annu Rev Genet. Vol. 33; 1999:351-397.
23. O'Brien T W. Properties of human mitochondrial ribosomes. IUBMB Life. Vol. 55; 2003:505-513.
24. Gaur R, Grasso D, Datta P P, et al. A single mammalian mitochondrial translation initiation factor functionally replaces two bacterial factors. Mol. Cell. Vol. 29; 2008:180-190.
25. Zhang Y, Spremulli L L. Roles of residues in mammalian mitochondrial elongation factor Ts in the interaction with mitochondrial and bacterial elongation factor Tu. J Biol. Chem. Vol. 273; 1998:28142-28148.
26. Hunter S E, Spremulli L L. Mutagenesis of glutamine 290 in Escherichia coli and mitochondrial elongation factor Tu affects interactions with mitochondrial aminoacyl-tRNAs and GTPase activity. Biochemistry. Vol. 43; 2004:6917-6927.
27. Yunis A A. Chloramphenicol toxicity: 25 years of research. Am J. Med. Vol. 87; 1989:44 N-48N.
28. Leach K L, Swaney S M, Colca J R, et al. The site of action of oxazolidinone antibiotics in living bacteria and in human mitochondria. Mol. Cell. Vol. 26; 2007:393-402.
29. Nagiec E E, Wu L, Swaney S M, et al. Oxazolidinones inhibit cellular proliferation via inhibition of mitochondrial protein synthesis. Antimicrob Agents Chemother. Vol. 49; 2005:3896-3902.
30. Mawji I A, Simpson C D, Hurren R, et al. Critical role for Fas-associated death domain-like interleukin-1-converting enzyme-like inhibitory protein in anoikis resistance and distant tumor formation. J Natl Cancer Inst. Vol. 99; 2007:811-822.
31. Buick R N, TILL JE, McCULLOCH EA. Colony assay for proliferative blast cells circulating in myeloblastic leukaemia. Lancet. Vol. 1; 1977:862-863.
32. Schimmer A D, Thomas M P, Hurren R, et al. Identification of small molecules that sensitize resistant tumor cells to tumor necrosis factor-family death receptors. Cancer Res. Vol. 66; 2006:2367-2375.
33. Tam E W Y, Feigenbaum A, Addis J B L, et al. A novel mitochondrial DNA mutation in COX1 leads to strokes, seizures, and lactic acidosis. Neuropediatrics. Vol. 39; 2008:328-334.
34. Heuser M, Argiropoulos B, Kuchenbauer F, et al. MN1 overexpression induces acute myeloid leukemia in mice and predicts ATRA resistance in patients with AML. Blood. 2007 Sep. 1; 110(5):1639-47.

1. A method of inducing cytotoxicity in a cancer cell comprising contacting the cancer cell with a glycylcycline.

2. A method for treating a subject with cancer comprising administering to the subject an effective amount of a glycylcycline.

3. (canceled)

4. (canceled)

5. A method of treating a subject with cancer according to claim 2 comprising: a) obtaining a test sample from a subject; b) determining a mitochondrial DNA copy number and/or a mitochondrial mass of the test sample; c) comparing the mitochondrial DNA copy number and/or mitochondrial mass of the test sample to a mitochondrial DNA copy number and/or a mitochondrial mass of a control, and d) administering tigecycline to the subject when the mitochondrial DNA copy number and/or the mitochondrial mass of the test sample is at least 2 fold increased compared to the mitochondrial DNA copy number and/or the mitochondrial mass of the control.

6. The method according to claim 2 wherein the cancer has at least 2 fold increased mitochondrial DNA copy number and/or mitochondrial mass compared to a control.

7. The method of claim 1, wherein the glycylcycline is tigecycline.

8. (canceled)

9. The method, of claim 2, wherein the cancer is a hematological cancer.

10. The method of claim 9, wherein the hematological cancer is a leukemia, a lymphoma or myeloma.

11. The method of claim 10, wherein the leukemia is AML, ALL, CLL or CML.

12. The method of claim 2, wherein the cancer is a solid tumour cancer.

13. The method of claim 12, wherein the solid tumour cancer is a lung, ovarian or prostate cancer.

14. The method of claim 2, wherein the glycylcycline, optionally tigecycline, administered is comprised in a composition, dosage or dosage form.

15. (canceled)

16. (canceled)

17. The method of claim 14, wherein the dosage form is selected from a solid dosage form and a liquid dosage form.

18. The method of claim 17, wherein the composition is administered by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

19. The method of claim 18, wherein the composition comprises an injectable dosage form.

20. The method of claim 17, wherein the composition is administered by intratumoral injection or intratumor vasculature injection.

21. The method of claim 17, wherein each unit dosage form comprises from about 100 mg to about 2000 mg, from about 100 mg to about 1500 mg, from about 100 mg to about 1000 mg, from about 100 mg to about 700 mg, from about 100 mg to about 500 mg, from about 100 mg to about 350 mg, from about 100 mg to about 300 mg or from about 100 mg to about 250 mg of a glycylcycline, for example tigecycline.

22. The method of claim 17, wherein each unit dosage form comprises about 20 to about 100 mg of an glycylcycline/kg body weight, about 30 to about 100 mg of an glycylcycline/kg body weight, about 40 to about 100 mg of an glycylcycline/kg body weight, or about 50 to about 100 mg of an glycylcycline/kg body weight of a subject in need of such treatment formulated into a solid oral dosage form, a liquid oral dosage form, or an injectable dosage form.

23. A method of identifying a subject likely to benefit from administration of a glycylcycline comprising:
obtaining a test sample comprising cancer cells from a subject;
determining a mitochondrial DNA copy number and/or a mitochondrial mass of the test sample; and
comparing the mitochondrial DNA copy number and/or the mitochondrial mass of the test sample to a mitochondrial DNA copy number and/or a mitochondrial mass of a control,
wherein the subject is identified as likely to benefit from administration of a glycylcycline when the test sample cancer cells have an at least 2 fold increased mitochondrial DNA copy number and/or mitochondrial mass compared to the control.

24. A kit comprising a glycylcycline and instructions and/or packaging materials for use in a method, according to claim 2.

25. A kit according to claim 24, wherein the glycylcycline is tigecycline.
